(19) United States
(12) Patent Application Publication
(10) Pub. No.: US 2004/0185475 A1
(43) Pub. Date: Sep. 23, 2004

(54) Methods for genotyping ultra-high complexity DNA

(75) Inventors: Manqiu Cao, Fremont, CA (US); Giulia Kennedy, San Francisco, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/740,230

(22) Filed: Dec. 17, 2003

Related U.S. Application Data
(60) Provisional application No. 60/454,090, filed on Mar. 11, 2003.
(60) Provisional application No. 60/453,930, filed on Mar. 11, 2003.
(60) Provisional application No. 60/496,539, filed on Aug. 20, 2003.
(60) Provisional application No. 60/443,499, filed on Jan. 28, 2003.

(51) Int. Cl. C12Q 1/68

(52) U.S. Cl. 435/6

(57) ABSTRACT

One aspect of the present invention provides methods and kits for genotyping ultra-high-complexity DNA. These include exemplary methods for increasing the size range of genomic DNA, which offers the benefit of increasing the complexity of the derived sample.

### RELATED APPLICATIONS

This application claims the priority of U.S. Provisional Application Serial Nos. 60/454,090; 60/453,930; 60/496,539; 60/228,253; 60/319,253; 60/319,685; 60/369,019; 60/389,745; 60/392,305; 60/392,406; 60/412,491; 60/392,305; 60/393,668; 60/389,701; 60/412,491; 60/417,190; 60/443,499; and U.S. patent application Ser. Nos. 09/766,212, 10/264,945, 10/316,629, 10/321,741, 09/916,135, and 10/316,517. All cited applications are incorporated herein by reference for all purposes.

### FIELD OF THE INVENTION

This application is related to nucleic acid analysis.

### BACKGROUND OF THE INVENTION

The ability to sample variation across entire genomes is central to mapping disease genes and understanding population history and evolution. Such studies are estimated to require analysis of 10,000-300,000 single-nucleotide polymorphisms (SNPs) in many individual DNA samples. In order to decrease the number of individual samples and maximize the efficiency of such analyses, it is desirable to sample ultra-high-complexity genomic DNA fractions and thereby gain maximum coverage of SNPs at a given time. The primary barrier to genotyping ultra-high complexity genomic fractions on arrays has been in the loss of signal-to-noise ratio. This results in low call rates and low accuracy.

### SUMMARY OF THE INVENTION

In one aspect of the invention, a large scale genotyping approach is provided. This approach is useful for genotyping at least 5000, 10,000, 50,000, 100,000 SNPs in complex DNA.

In some embodiments, the methods begin with in silico prediction of SNPs residing in desired genomic fractions and synthesis of these SNP-containing fragments onto high-density microarrays. Following biochemical fractionation that mirrors the in silico fractionation, target is hybridized to microarrays and SNPs are genotyped by allele-specific hybridization.

In some embodiments, the method includes the steps of processing a genomic DNA sample in fewer than 2, 5, 10, 15 or 20 reaction vessels to obtain a nucleic acid sample; and hybridizing the nucleic acid sample to a collection of at least 10,000 different oligonucleotide probes to determine the genotypes of greater than 5,000, 10,000, 50,000, 100,000 SNPs. As used herein, the term “reaction vessel” refers to any suitable device suitable for hosting a chemical reaction. Examples of suitable vessels include microtiter plate wells, test tubes (other suitable containers), eppendorf tubes, microfluidic reaction chambers, etc.

The oligonucleotide probes may be immobilized on a substrate to form microarrays or immobilized to a collection of beads.

In another aspect of the invention, methods for analyzing very high complexity DNA samples are provided. As shown in FIG. 9, an exemplary method was used to analyze a nucleic acid sample derived from a genomic sample. The nucleic acid sample represented up to approximately 500 Mbases of genomic DNA. The nucleic acid sample was hybridized with a WGSA genotyping array (Affymetrix, Santa Clara, Calif.). The resulting hybridization patterns were analyzed to generate SNP calls. The calls are highly accurate (call rate of approximately 90% or higher) and have a very high concordance.

In some embodiments, the method includes the step of processing a genomic DNA sample in a single reaction to obtain a nucleic acid sample enriched for 500-2000 bp sized fragments; and hybridizing 40-80 (40, 60, 80) μg of the enriched nucleic acid sample to a collection of probes to determine the genotypes of greater than approximately 200-500 Mbp (mega base pairs) of genomic DNA.

In another aspect of the invention, methods for controlling the size range of the amplified DNA fragments are provided. By controlling the size range, one can adjust the complexity of the sample and thus the number of SNPs that can be interrogated. The methods may include employing specific PCR conditions or specific DNA polymerases. In some embodiments, methods are provided to preferentially amplify DNA fragments ranging from approximately 500-2000 bp. In one embodiment, Pfx polymerase is used and PCR conditions are optionally modified to achieve this size range. Increasing size range offers the benefit of increasing the complexity of the derived sample.

In another aspect of the invention, methods are provided for genotyping at least 200 megabases of genomic DNA for SNP genotypes. In some embodiments, a high complexity genomic DNA sample is processed using the methods of the invention for controlling the size range of amplified fragments. The amplified fragments are interrogated for SNP genotypes using high density oligonucleotide probe arrays.

In yet another aspect of the invention, methods for enzymatic sample preparation design, microarray design and data analysis are also provided.

### BRIEF DESCRIPTION OF THE FIGURES

The accompanying drawings, which are incorporated in and form a part of this specification, illustrate embodiments of the invention and, together with the description, serve to explain the embodiments of the invention:

FIG. 1 Fragment Selection by PCR (FSP). Digestion of genomic DNA with a restriction enzyme (e.g. BglII), results in fragments of various sizes (black), including fragments 400-800 bp long (red). Adaptors are ligated to all size fragments, but only those fragments in the 400-800 bp size range are amplified. The amplified target is fragmented and labeled and hybridized to synthetic DNA microarrays.

FIG. 2 Hybridized chip images a. Microarray hybridized to reduced complexity (˜4×10⁷ bp) biotin-labeled DNA b. Microarray hybridized with biotin-labeled human genomic DNA (3×10⁹ bp). Signals from hybridization controls are detected. c. SNP miniblock showing hybridization of FSP target in three individuals, demonstrating the three possible genotypes; AA (left), AB (middle) and BB (right). Probes are synthesized as perfect match (PM) 25-mers, and as one-base mismatches (MM) in the center. Probes for both A and B alleles, on both sense and antisense strands are synthesized, for a total of 56 probes per SNP miniblock.

FIG. 3 Cluster visualization of SNPs. Relative allele signal (RAS) is calculated for each sample on both strands and plotted in two dimensions, demonstrating various types of clustering properties: a. SNP with ideal clustering properties; b. SNP forming 3 distinct clusters in the sense, but not antisense, dimension; c. Poorly clustering SNP; d. This SNP forms two well-separated and tight clusters; genotyping of additional samples may reveal instances of the minor allele homozygote (in this case, BB).

FIG. 4 Inter-SNP distances on Golden Path. The SNP map positions were determined by TSC on the April 2002 release of the Golden Path (NCBI Build 29). The distances between markers, in kb, are plotted as a frequency distribution. The cumulative % of markers is indicated by the dotted line

FIG. 5 Distribution of heterozygosity in three populations. The frequency of heterozygotes for each SNP was determined in 3 populations and plotted as a distribution across 10 bins, plus an additional category for SNPs that showed zero heterozygotes in that population, ie monomorphic SNPs (leftmost bars).

FIG. 6 Percentage ancestral allele as a function of allele frequency in three populations. Genotypes were determined for chimp and gorilla and the percent A allele was calculated for each frequency bin. The “A” allele for each SNP was determined alphabetically

FIG. 7 Comparison of PCR amplification profiles using Taq polymerase and Pfx polymerase. Genomic DNA was digested with AflII or BclI or BglII and amplified with Taq polymerase (Panel A) or Pfx polymerase (Panel B). Fragment sizes were measured against a standard DNA ladder.

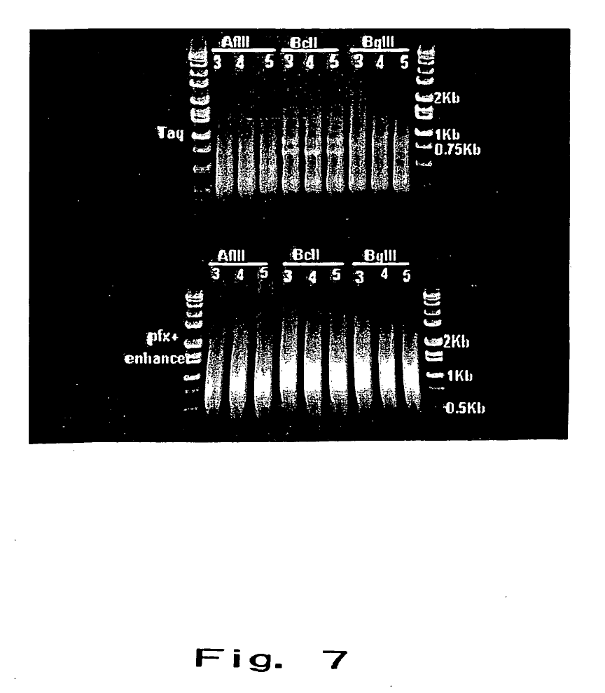

FIG. 8 Comparison of PCR amplification profiles using Taq polymerase and Pfx polymerase. Genomic DNA was digested with EcoRI or NcoI or SacI and amplified with Taq polymerase (Panel A) or Pfx polymerase (Panel B). Fragment sizes were measured against a standard DNA ladder.

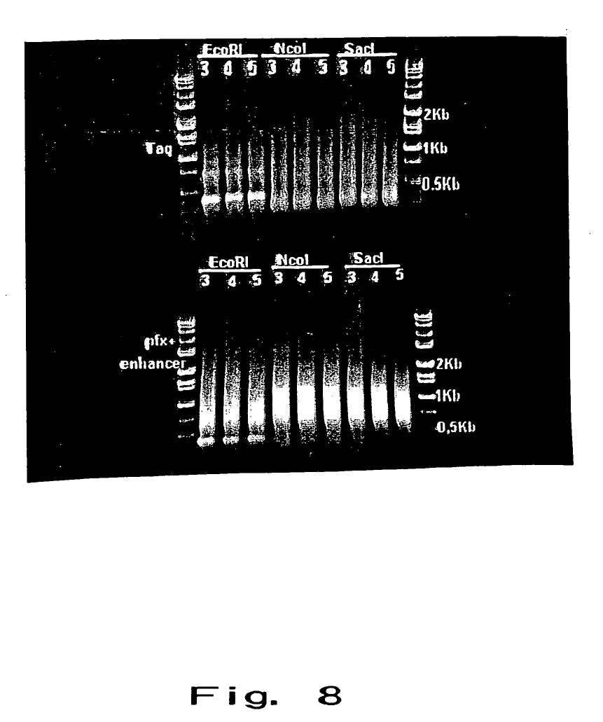

FIG. 9 Percentage concordance or call as a function of complexity (Mb).

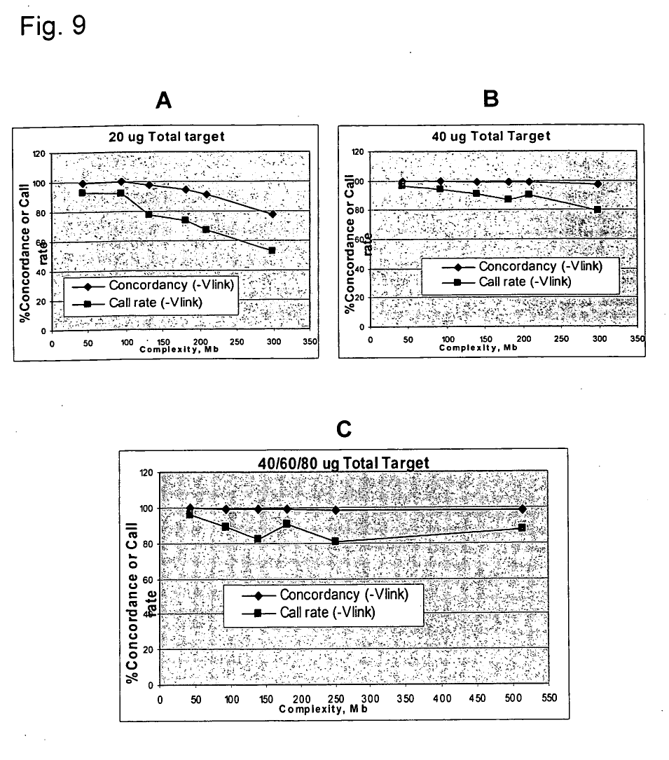

### DETAILED DESCRIPTION OF THE EMBODIMENTS OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the Affymetrix website.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, New York, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No 4,437,975), arbitrarily primed polymerase chain reaction (AP-PCR) (U.S. Pat. No 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P. N. A. S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. patent application Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

II. Glossary

The following terms are intended to have the following general meanings as there used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point™ fro the specific sequence at s defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Effective amount refers to an amount sufficient to induce a desired result.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

III. Methods for Genotyping Ultra-High Complexity DNA

In one aspect of the invention, methods are provided for large scale genotyping. In some embodiments, a genomic fractionation strategy is provided to leverage the large numbers of SNPs deposited in public databases. In preferred embodiments, methods avoid the use of individual SNP-specific primers. The reduction and amplification are highly reproducible, capturing a majority of the same SNPs across many samples.

In one embodiment, in order to take advantage of the large numbers of SNPs already discovered, methods are provided to recapitulate fractionation schemes used by the various genome centers in The SNP Consortium (“TSC”) for discovering SNPs. Protocols used by TSC include digestion of genomic DNA from a pool of ethnically diverse individuals with one of several restriction enzymes, followed by gel electrophoresis to isolate fragments within a desired size range (Altshuler, D., Pollara, V. J., Cowles, C. R., Van Etten, W. J., Linton, L., Baldwin, J., & Lander E. S. A SNP map of the human genome generated by reduced representation shotgun sequencing. Nature. 2000 Sep. 28; 407(6803):513-6). DNA from these gel fractions was extracted and used to construct plasmid libraries, from which individual clones were sequenced and SNPs discovered (Altshuler, D., Pollara, V. J., Cowles, C. R., Van Etten, W. J., Linton, L., Baldwin, J., & Lander E. S. An SNP map of the human genome generated by reduced representation shotgun sequencing. Nature. 2000 Sep. 28;407(6803):513-6). By choosing the same restriction enzymes and size fractions used by TSC, target preparation would be enriched for high-quality, validated, publicly-available SNPs.

In some preferred embodiments, the method begins with in silico prediction of SNPs residing in desired genomic fractions and synthesis of these SNP-containing fragments onto high-density microarrays. In order to genotype as many TSC SNPs as possible on the fewest numbers of arrays, the arrays can be designed to interrogate only those SNPs predicted to be amplified by the biochemical assays.

Completion of the draft human genome sequence made it possible to conduct in silico digests of total genomic DNA, identify the desired size fragments, and predict which SNPs should be present on those fragments. Fragments containing repetitive sequences within the tiled region are excluded; these represented about 25-30% of TSC SNPs.

In one aspect of the invention, methods are provided for analyzing high complexity genomic DNA. In some embodiments, samples representing at least 40, 100, 200, 300, 400, 500 Mbp (mega base pairs) of genomic DNA can be analyzed. The methods can be used to genotype at least 10,000, 50,000, 75,000, 100,000 or more SNPs with a single tube assay and optionally, single hybridization.

In another aspect of the invention, sample preparation, hybridization and data analysis methods are provided. The hybridization and data analysis methods are described in various sections of this specification and cited references. In some exemplary embodiments of the sample preparation methods, a genomic DNA sample is processed in a single reaction to obtain a nucleic acid sample enriched for 500-2000 bp sized fragments, and subsequently, up to 40 μg of the enriched nucleic acid sample is hybridized to a collection of probes to determine the genotypes of greater than 200 Mbp (mega base pairs) of genomic DNA. In some preferred embodiments, up to 80 μg of the enriched nucleic acid sample is hybridized, enabling genotyping of nearly 500 Mbp of genomic DNA. These results were achieved at similar concordance/ call rates or accuracies. Concordance rates were generally found to increase when the amount of target nucleic acid sample was increased.

In an example, a series of 11 arrays containing sequence from 71,931 unique SNPs present in three different genomic subfractions (EcoRI, BglII and XbaI) were synthesized. A total of 56 probes were synthesized for each SNP. For each SNP, probes (25-mers) were synthesized, spanning seven positions along both strands of the SNP-containing sequence, with the SNP position in the center, (position zero) as well as at −4, −2, −1, +1, +3, +4. Four probes were synthesized for each of the 7 positions: a perfect match (PM) for each of the two SNP alleles (A, B) and a one-base central mismatch (MM) for each of the two alleles, as described previously. Normalized discrimination, calculated as (PM−MM)/(PM+MM) is a measure of sequence specificity, and is used in the detection filter of the genotype calling algorithm (Liu, W. -M., Mei, R., Bartell, D. M., Di, X., Webster, T. A. and Ryder, T. (2001) Rank-based algorithms for analysis of microarrays. In Microarrays: Optical technologies and Informatics. Edited by Bittner, M. L., Chen, Y., Dorsel, A. N. and Dougherty, E. R. Proc. SPIE, 4266, 56-67). Probes were synthesized for both sense and antisense strands, for a total of 56 probes per SNP. Following biochemical fractionation that mirrors the in silico fractionation, target is hybridized to arrays and SNPs are genotyped by allele-specific hybridization.

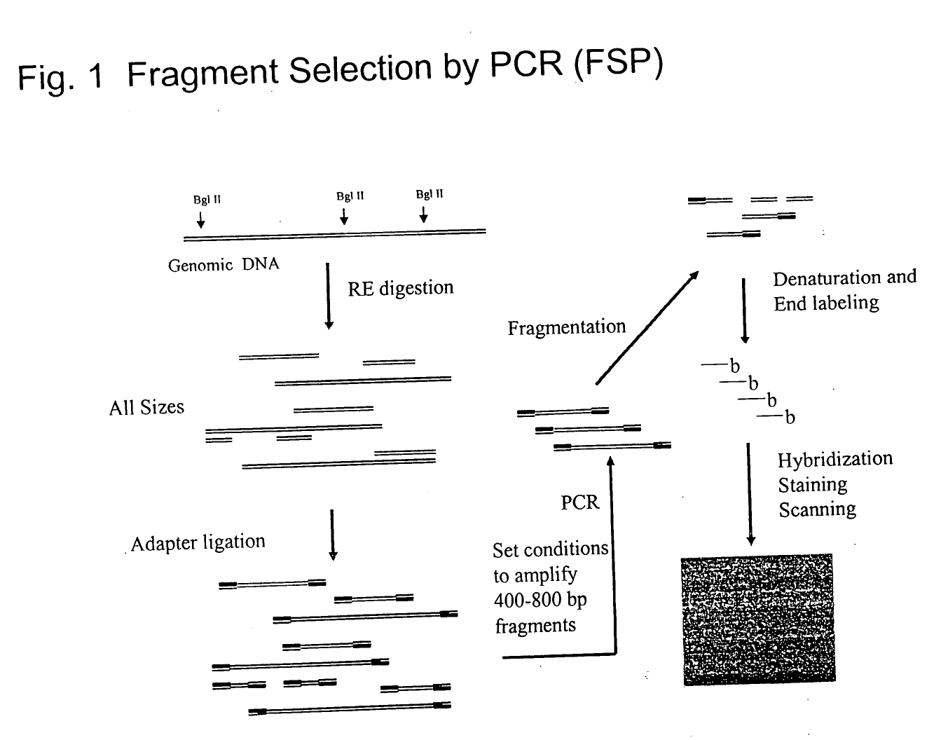

In another aspect of the invention, a biochemical fractionation method, called “Fragment Selection by PCR” or FSP, is provided. The FSP method is illustrated in FIG. 1. Total genomic DNA is digested with one of several restriction enzymes and ligated to the digested DNA with adaptors recognizing the cohesive four bp overhangs. All fragments resulting from restriction enzyme digestion, regardless of size, are substrates for adaptor ligation. A generic primer, which recognizes the adaptor sequence, is used to amplify ligated DNA fragments.

In some embodiments, the PCR reaction conditions are optimized to selectively and reproducibly amplify fragments in a particular size range, for example, at least 30%, 40%, 50%, 60%, 70%, 80% and 90% (enriched) of the resulting nucleic acids are in the 500-2000 bp size range in the example, thereby achieving both fractionation of the genome and maximization of SNP content (such as the TSC SNP content). In some embodiments, DNA polymerases of different processivity and fidelity may be selected to achieve the size enrichment. For example, AccuPrime™ or Platinum®Pfx (Invitrogen, Carlsbad, Calif.) may be used. One of skill in the art would appreciate that the invention is not limited to these specific enzymes.

The PCR reaction can be carried out in the exemplary reaction:

| Reagent | Volume (in μl) | |
|---|---|---|
| H₂O | 55 | |
| 10× Pfx buffer, Invitrogen | 10 | |
| 10× PCR enhancer, Invitrogen | 10 | |
| dNTP, 25 mM each | 1 | |
| 50 mM MgSO₄ | 2 | |
| Primer 001-FivePR, 10 uM | 10 | |
| DNA template, 2.5 ng/ul | 10 | |
| Pfx, 2.5 u/μl, Invitrogen | 2 | |
| Total | 100 | μl |

The following conditions may be employed for the PCR reaction:

| Temperature | Time |
|---|---|
| 94° C. | 3 min |
| 35 cycles of | 15 sec |
| 94° C. | |
| 60° C. | 20 sec |
| 68° C. | 40 sec |
| 68° C. | 7 min |
| 4° C. | ∞ |

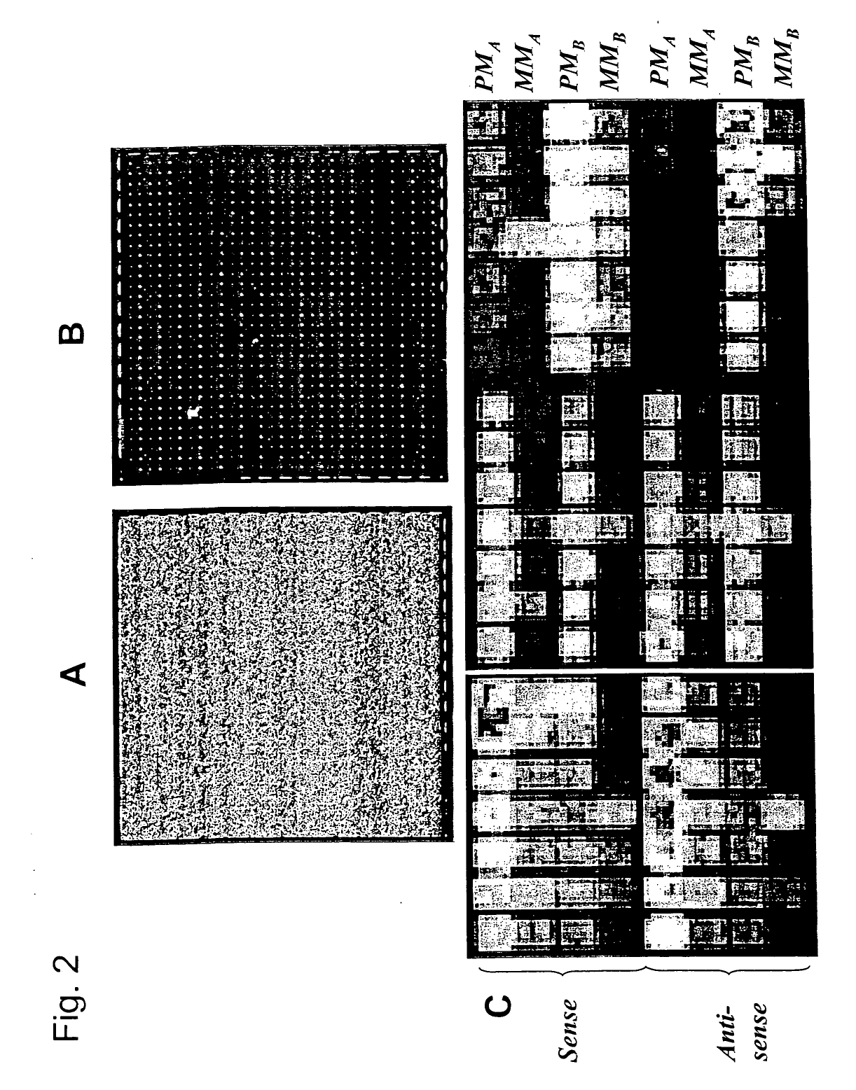

In an exemplary embodiment, targets generated by FSP were labeled and hybridized to the arrays. Each fraction represents approximately 4×10⁷ bp of genomic DNA (estimation of complexity is affected by several factors: accuracy of genome sequence used for in silico fractionations, efficiency of adaptor ligation and amplification; the theoretical value for complexity based on the draft human genome sequence (April 2001 release) was calculated and uniform amplification of target fragments was assumed). An image of a representative array hybridized with one fraction shows robust signal intensities (FIG. 2A). In contrast, hybridization of total human genomic DNA (3.2×10⁹ bp) results in low signals (FIG. 2B), a substantial portion of which is noise. A close-up view of a SNP “block” hybridized with DNA from three different individuals representing all three genotypes is shown in FIG. 2C. Hybridization signals which allow interpretation of genotypes are clearly visible by eye, demonstrating the feasibility of our generic approach.

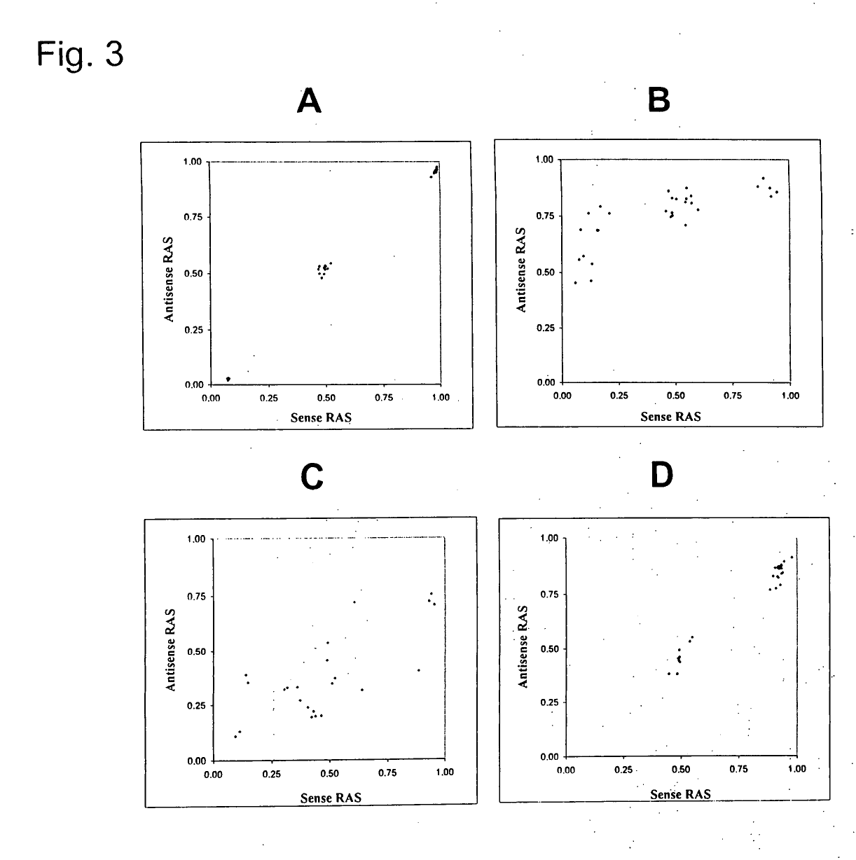

In another aspect of the invention, an automated scoring process for calling genotypes is provided. In the example, the training data was derived from 30 ethnically diverse DNA samples (Samples used in the training set included 24 individuals from the polymorphism discovery panel (PD1-24), along with 6 unrelated CEPH individuals, all available through the Coriell Institute for Medical Research as part of the National Institute of General Medical Sciences Human Genetic Mutant Cell Repository. Relative allele signal (RAS) values for each SNP on both sense and antisense strands were calculated and plotted for all 30 individuals in two dimensions (RAS is calculated as the median of the ratios Ai/(Ai+Bi), where Ai and Bi are signals of A and B alleles of the ith probe quartet). Some SNPs show three clearly defined clusters (FIG. 3A), while others show more diffuse clusters (FIG. 3B), or no clear clusters at all (FIG. 3C). For those SNPs having lower minor allele frequencies, the genotypes fall into only two clusters, with the minor allele homozygote cluster being absent (FIG. 3D). Following graphic visualization of clusters derived from RAS values in two dimensions, an algorithm was developed to classify these points into two or three clusters and evaluate the quality of classification with the average silhouette width, s (The silhouette width is a relative measure of the difference between the distance of a data point to the nearest neighbor group and the distance of the data point to other data points in the same group. Silhouette widths range from −1 to 1; the larger the silhouette width, the better the classification from a clustering point of view (Rousseeuw, P. J. (1987) Silhouettes: a graphical aid to the interpretation and validation of cluster analysis, J. Comput. Appl. Math. 20, 53-65)). The algorithm includes a signal detection filter based on Wilcoxon's signed rank test (Liu, W. M., Mei, R., Bartell, D. M., Di, X., Webster, T. A. and Ryder, T. (2001) Rank-based algorithms for analysis of microarrays. In Microarrays: Optical technologies and Informatics. Edited by Bittner, M. L., Chen, Y., Dorsel, A. N. and Dougherty, E. R. Proc. SPIE, 4266, 56-67), classification using a modification of partitioning around medoids (PAM) (Kaufman, L. and Rousseeuw, P. J. (1987) Clustering by means of medoids, in Statistical Data Analysis based on L1 norm, edited by Y. Dodge, Amsterdam: Elsevier, pp.405-416. Kaufman, L. and Rousseeuw, P. J. (1990). Finding Groups in Data: An Introduction to Cluster Analysis, New York: John Wiley & Sons, Inc.) and the computation of several quality scores). As s approaches 1.0, clusters are tight and well-separated, while low values of s, e.g. <0.5, are derived from poorly clustering SNPs.

A series of heuristics for ranking the SNPs according to their clustering properties were also used (SNPs were selected based on the following criteria: those that formed three clusters with s>0.7, showed separation of RAS medians between clusters >0.2, and 27 out of 30 samples passed the detection filter). In the example, of the 71,931 SNPs assessed in this experiment, −20% or 14,548 met the most stringent criteria. Only SNPs that formed three clusters were scored. Following clustering, it was determined that boundaries around the clusters for the purposes of assigning incoming points to one of the clusters, i.e., making genotype calls. Several genotype calling methods were developed. The method used in this example assigns a center point for each cluster. The coordinates of the center are the sense and antisense medians of all points in a cluster. The genotype call boundary was determined by the Euclidian distance to the center, and the call zone is then restricted to 80% of that distance); therefore many SNPs that formed only two good clusters did not meet the cut-off criteria. When the training set was increased from 30 to 108 individuals, the percentage of SNPs meeting the stringent criteria increased, suggesting that many of the SNPs form only two clusters due to lower minor allele frequencies, i.e. the minor allele homozygote was not observed.

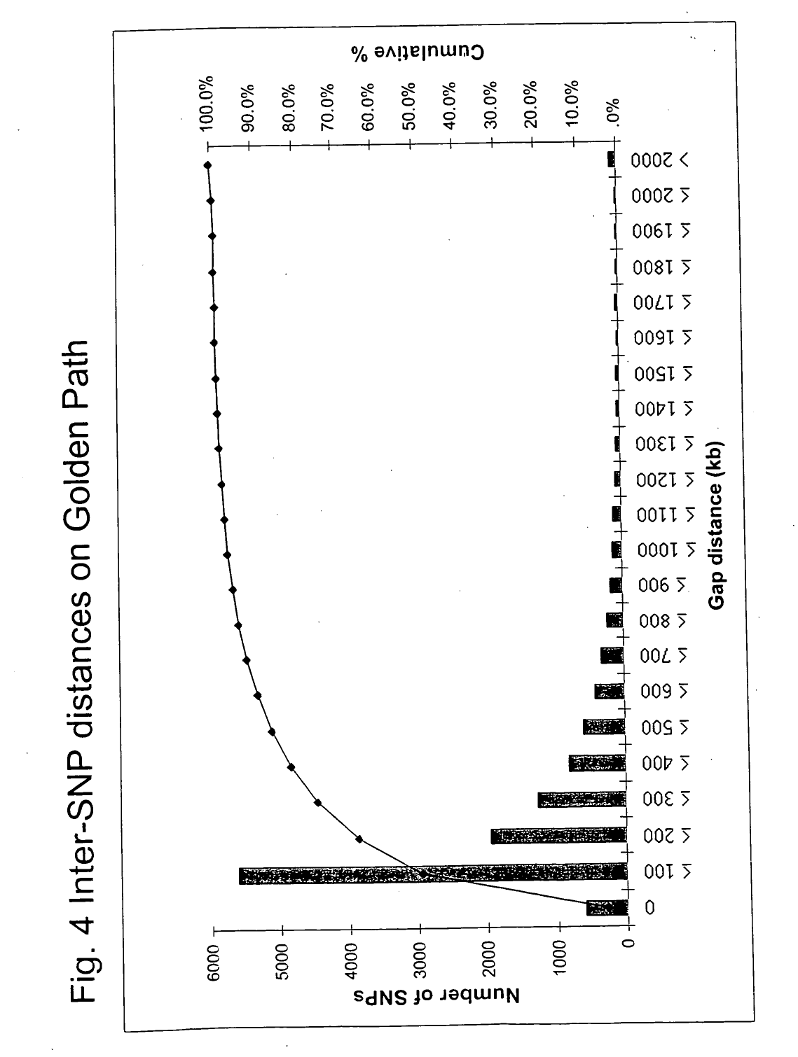

In the example, the mean and median heterozygosity of the 14,548 markers is 0.386 and 0.421, respectively (theoretical maximum=0.50), indicating that these markers should be highly informative in a variety of ethnic populations studied here. All markers were mapped on the Golden Path human genome sequence by TSC. The distribution of inter-SNP distances between markers is shown in FIG. 4; the mean and median intermarker distances are 174 kb and 80.8 kb, respectively. Of these markers, 5058 are spaced at distances of 50 kb or less; 3868 are spaced at distances of 25 kb or less. This density allows mapping in familial linkage studies and is predicted to capture some proportion of linkage disequilibrium in the genome.

Genetic studies typically involve genotyping hundreds of samples, thus all genotyping methods must interrogate SNPs reproducibly across DNA samples. In the example, the average genotype call rate is 95.1%±1.2%, demonstrating a high level of reproducibility (Reproducibility was determined on a set of 38 Caucasian samples, genotyped as incoming data on clusters defined by the N=30 training set. The percentage of successful genotype calls (call rate) was averaged over 38 samples and ranged from 91.5-97.3%). The accuracy of our genotype calls was determined in two ways: through the use of genotypes obtained by independent genotyping methods, and by dideoxynucleotide sequencing of discordant genotype calls. The accuracy of genotypes in this example was determined to be >99.5% (reference genotypes for approximately 900 SNPs assayed were obtained using single-base extension (SBE) technology and compared these genotypes to those generated by Whole Genome Assay. A concordance rate of 99.1 % was found for these markers over 38 samples (total of 33,111 calls compared). Ten SNPs accounted for >50% of the 311 discordant genotypes. De novo nucleotide sequence for these 10 SNPs across individuals exhibited discordant genotypes, and it was found that Whole Genome Sampling Assay (WGSA) genotype calls were concordant with sequence data 44% of the time. Thus, the accuracy of WGSA genotype calls is most likely >99.5%. Genotypes for 65 SNPs across 7 individuals were compared with data derived from high-resolution scanning of chromosome 21. Of 287 calls compared between the two datasets, there was only one discordant genotype (i.e. concordance rate=99.6%). Additional confidence in the accuracy of our genotype calls was obtained indirectly by examining genomic DNA isolated from two complete hydatidiform moles (CHM). These products of abnormal conception arise from the fertilization of an empty ovum by a single sperm, resulting in complete duplication of the haploid paternal genome. Genotypes are expected to be homozygous for all markers (Fan, J. -B., Surti, U., Taillon-Miller, P., Hsie, L., Kennedy, G. C., Hoffner, L., Ryder, T., Mutch, D. G., Kwok, P. -Y. (2002) Paternal Origins of Complete Hydatidiform Moles Proven by Whole Genome Single-Nucleotide Polymorphism Haplotyping. Genomics, 79: 58-62). Both tumors showed 0.4% heterozygosity, consistent with expectations of a completely duplicated haploid genome, while a control sample of normal placenta showed 35.3% heterozygosity.

In the example, SNPs present in multiple enzyme fractions were also studied and tiled on two or more arrays. Of the 205 SNPs synthesized on two or more arrays and captured by different enzyme fractions, the concordance rate for genotype calls was 99.5% across 30 individuals.).

In the example, embodiments of the methods of the invention were used to rapidly determine the allele frequencies of >13,647 SNPs in DNA from 60 unrelated individuals comprising three human populations: Caucasian, African-American and Asian (Samples from the three populations (denoted TSC DNA panels) are available from Coriell on the Cold Spring Harbor Laboratory website. A comparison of the allele frequencies derived from a set of 20 Caucasians versus a set of 38 Caucasians shows a high correlation (R²=0.96), indicating that sampling of 20 individuals provides reasonably stable estimates of allele frequencies for these SNPs in that population. Furthermore, allele frequencies for 313 of our SNPs were also determined by TSC as part of the allele frequency project (AFP) and these allele frequencies agree well with ours (A total of 313 SNPs overlapped our data set and that of TSC allele frequency project (AFP). A scatter plot of the allele frequencies in the two data sets showed a correlation coefficient R2=0.90).

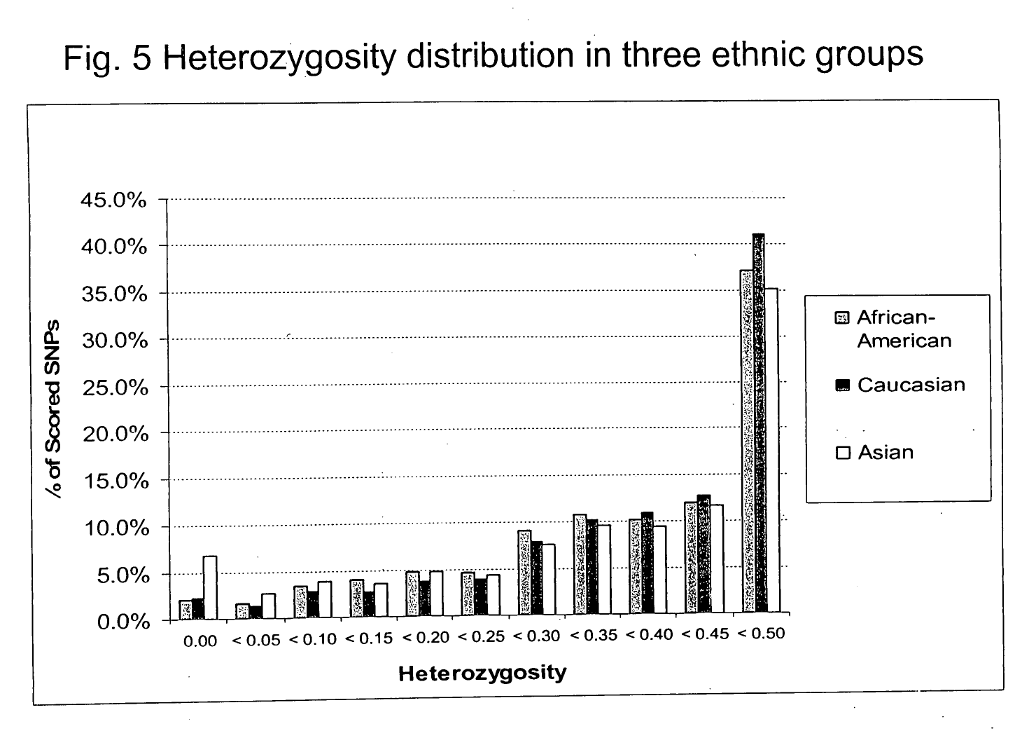

Of the 13,647 SNPs interrogated, the vast majority were polymorphic in all three populations. This is consistent with expectations, as the training set consisted of an ethnically diverse panel of individuals. The distribution of marker heterozygosity in the three populations was also determined (FIG. 5). The mean heterozygosity of the markers was 0.366, 0.358 and 0.373 in the African-American, Caucasian and Asian samples, respectively. 1 In this analysis, there were 343, 535 and 1219 markers in the African-American, Caucasian and Asian samples, respectively, which were monomorphic (i.e. zero heterozygosity). Of these, 100 were monomorphic in both African-Americans and Asians (but not Caucasians), 81 were monomorphic in African-Americans and Caucasians (but not in Asians) and 236 were monomorphic in both Asians and Caucasians (but not African-Americans).

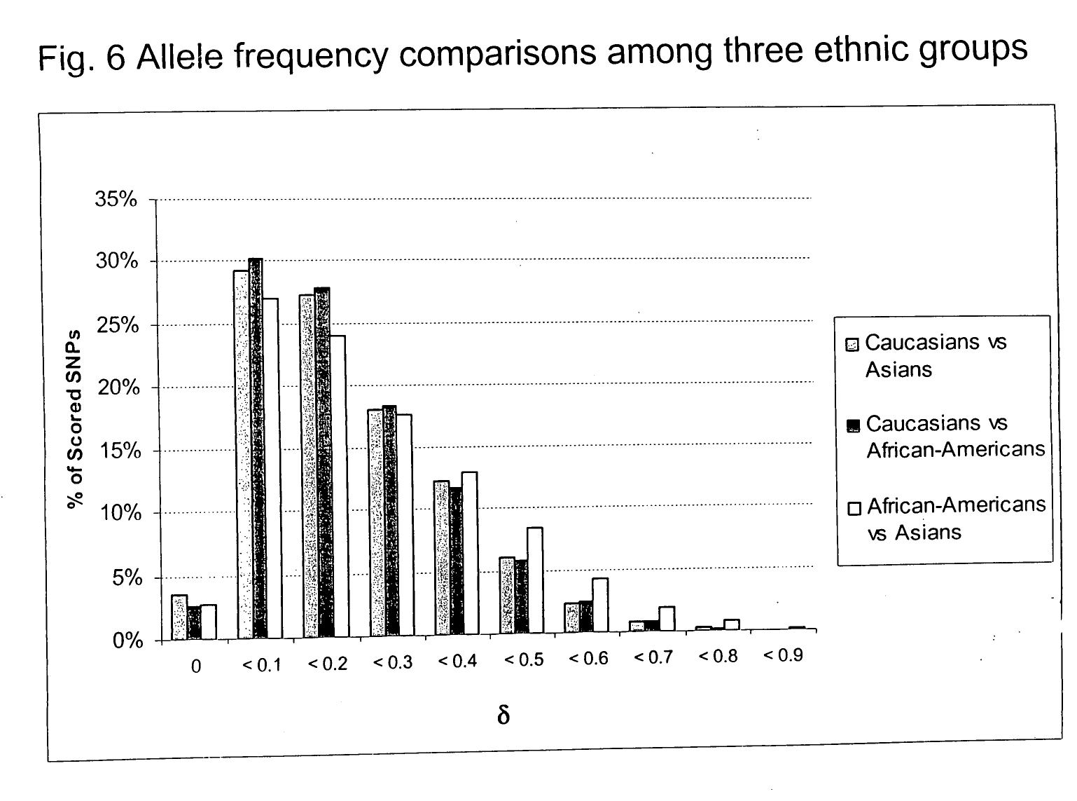

SNPs are “mutations” that have arisen once during evolution; to determine which of the two alleles represents the ancestral state, genotypes on chimpanzee and gorilla genomic DNA samples were determined. Chimpanzee and gorilla DNA differs from human by 1.5% and 2.1%, respectively (Hacia J G. Genome of the apes. Trends Genet 2001 Nov., 17(11):637-45)). Synthetic arrays have been used previously to score chimpanzee and gorilla genotypes on human SNPs (Hacia J G, Fan J B, Ryder O, Jin L, Edgemon K, Ghandour G, Mayer R A, Sun B, Hsie L, Robbins C M, Brody L C, Wang D, Lander E S, Lipshutz R, Fodor S P, Collins F S. Determination of ancestral alleles for human single-nucleotide polymorphisms using high-density oligonucleotide arrays. Nat Genet 1999 Jun;22(2): 164-7). Our results indicate that chimpanzee and gorilla genotypes can be called on 77.1% and 71.8% of the human SNPs, respectively (Table 1). The overwhelming majority of markers are homozygous in both great ape species (Table 1), consistent with the recent evolutionary history of SNPs. There are a small number of heterozygous SNPs that may represent shared (and thus very ancient) polymorphisms, however data from a larger number of great apes is necessary to assess Hardy-Weinberg equilibrium of these markers. Ancestral alleles were only assigned to SNPs that met the following criteria: SNPs that were homozygous in both chimpanzee and gorilla, and that gave the same genotype call in both species. A total of 8386 SNPs were assigned. The distribution of the chimpanzee and gorilla (i.e. ancestral) alleles was plotted as a function of SNP allele frequency in three human populations and found in each case a strong positive correlation; the higher the SNP allele frequency, the higher the proportion of the ancestral allele (FIG. 6).

The slopes of the Caucasian and Asian populations are 0.62 and 0.52, respectively. These data indicate that in these two populations the ancestral allele is not always the most frequent allele; i.e. about 20% of the time, the newer allele has become more frequent in these populations, consistent with previous studies. In contrast, the slope of the curve in African-Americans is 0.97, indicating a nearly one-to-one correlation between ancestral state and allele frequency. In this population, regardless of relative allele frequency, the most frequent allele is almost always the ancestral allele, contrary to theoretical predictions.

The example shows the simultaneous genotyping of more than 10,000 SNPs. This approach can be used to genotype greater than 1000, 5000, 10,000, 50,000, 100,000, 200,000, or 300,000 SNPS. To genotype additional SNPs, additional restriction enzyme fractions may be used, regardless of whether size selection is accomplished through FSP or by other means. For example, the Sanger Center discovered >65,000 SNPs from Nsi-digested genomic DNA fragments 0.9-1.4 kb in size. Arrays containing 50,458 of these SNPs were synthesized. Target from 30 individuals using gel excision was prepared, and similar rates of SNP capture were found. With the Whole Genome Sampling Assay (WGSA) approach, one can use increasing numbers of enzyme fractions to genotype large numbers of SNPs and approach ultra-high genome mapping densities.

The exemplary generic approach requires only 1 restriction-enzyme-specific oligonucleotide for each genomic subfraction, plus one generic oligonucleotide that amplifies all SNPs. The interrogation of 71,931 SNPs in the present study required only four primers. Furthermore, a single microarray can interrogate simultaneously >10,000 SNPs by reducing the number of probes per SNP; such reduction can be achieved without loss of accuracy. Our approach not only scales to larger numbers of SNPs, but scales to other complex organisms as well. As draft genome sequencing is completed for other genomes such as mouse, a SNP discovery effort mirroring that of TSC, namely the use of restriction enzyme and size fractionation, is desirable. Implementation of these protocols for discovery of SNPs in complex organisms will enable immediate use of Whole Genome Sampling Assay technology and thus facilitate acceleration of genetic studies in model organisms.

In addition to the initial population studies reported here, the tools can now be applied across a variety of other scientific disciplines to address many pressing genetic questions, especially those requiring a dense set of markers spaced across the genome. For example, with this technology, it is feasible to create high-resolution haplotype maps (Gabriel S B, Schaffner S F, Nguyen H, Moore J M, Roy J, Blumenstiel B, Higgins J, DeFelice M, Lochner A, Faggart M, Liu-Cordero S N, Rotimi C., Adeyemo A, Cooper R, Ward R, Lander E S, Daly M J, Altshuler D. The structure of haplotype blocks in the human genome. Science 2002 Jun. 21; 296(5576):2225-9), to rapidly determine allele frequencies in other geographic populations, and to identify regions of LD across the genome, all at unprecedented resolution.

TABLE 1

| | Human | Chimp | Gorilla |
|---|---|---|---|
| Number SNPs called A | 4401 | 5475 | 5061 |
| Number SNPs called B | 4431 | 5495 | 5156 |
| Number SNPs called AB | 4731 | 256 | 238 |
| Number No Calls | 995 | 3332 | 4103 |
| Total Calls | 13563 | 11226 | 10455 |
| Number Attempted Calls | 14558 | 14558 | 14558 |
| CallRate | 93.20% | 77.10% | 71.80% |
| % A | 32.40% | 48.80% | 48.40% |
| % B | 32.67% | 48.95% | 49.32% |
| % AB | 34.88% | 22.80% | 2.27% |

### CONCLUSION

It is to be understood that the above description is intended to be illustrative and not restrictive. Many variations of the invention will be apparent to those of skill in the art upon reviewing the above description. Therefore, it is to be understood that the scope of the invention is not to be limited by the specific embodiments.

What is claimed is:

1. A method for analyzing genomic DNA, comprising:
processing a genomic DNA sample in a single reaction to obtain a nucleic acid sample enriched for 500-2000 bp sized fragments; and
hybridizing at least 40 μg of the enriched nucleic acid sample to a collection of oligonucleotide probes to determine the SNP genotypes of greater than 200 mega base pairs of genomic DNA.

2. The method of claim 1 wherein the at least 40 μg is at least 60 μg.

3. The method of claim 2 wherein the at least 60 μg is at least 80 μg.

4. The method of claim 3 wherein the at least 80 μg is at least 150 μg.

5. The method of claim 4 wherein the at least 150 μg is at least 200 μg.

6. The method of claim 5 wherein the probes are immobilized on a substrate to form a microarray.

7. The method of claim 5 wherein the probes are immobilized on beads.

8. The method of claim 1 wherein the processing comprises DNA amplification using a polymerase that possesses high processive enzyme.

9. The method of claim 8 wherein the high processive enzyme is a Pfx DNA polymerase or an equivalent.

10. A method for analyzing genomic DNA comprising:
obtaining a nucleic acid sample derived from said genomic DNA, wherein said nucleic acid sample represents at least 200 Mbases of said genomic DNA;
hybridizing said nucleic acid sample to an oligonucleotide probe array; and
analyzing the hybridization pattern to determine genomic information in said genomic DNA.

11. The method of claim 10 wherein said genomic information is SNP genotypes.

12. The method of claim 11 wherein said analyzing comprises analyzing the hybridization pattern to determine at least 25,000 SNPs.

13. The method of claim 12 wherein said nucleic acid sample represents at least 500 Mbases of said genontic DNA.

14. The method of claim 13 wherein said analyzing comprises analyzing said hybridization pattern to determine at least 75,000 SNPs.

15. The method of claim 14 wherein said analyzing comprises analyzing said hybridization pattern to determine at least 100,000 SNPs.

16. The method of claim 10 wherein the obtaining comprises performing a WGSA (FSP) assay using a polymerase that possesses a high processive enzyme activity.

17. The method of claim 16 wherein the high processive enzyme is a Pfx DNA polymerase or an equivalent.
